(19) United States
(12) Patent Application Publication
(10) Pub. No.: US 2026/0151654 A1
(43) Pub. Date: Jun. 4, 2026

(54) SYSTEM FOR NERVE MODULATION AND INNOCUOUS THERMAL GRADIENT NERVE BLOCK

(71) Applicant: Boston Scientific Scimed, Inc., Maple Grove, MN (US)

(72) Inventors: Roger N. Hastings, Naples, FL (US); Mark L. Jenson, Greenfield, MN (US)

(73) Assignee: Boston Scientific Scimed, Inc., Maple Grove, MN (US)

(21) Appl. No.: 19/455,280

(22) Filed: Jan. 21, 2026

Related U.S. Application Data
(63) Continuation of application No. 16/524,303, filed on Jul. 29, 2019, now Pat. No. 12,558,576.
Application No. 16/524,303 is a continuation of application No. 14/032,553, filed on Sep. 20, 2013, now Pat. No. 10,398,464.
(60) Provisional application No. 61/704,169, filed on Sep. 21, 2012.

(51) Int. Cl. A61N 7/02; A61B 17/32; A61N 7/00

(52) U.S. Cl. CPC A61N 7/022; A61B 17/320068; A61N 2007/0026; A61N 2007/003; A61N 2007/0043; A61N 2007/0078

(57) ABSTRACT

Systems for nerve and tissue modulation are disclosed. An example system may include a first elongate element having a distal end and a proximal end and having at least one nerve modulation element disposed adjacent the distal end. The nerve modulation element may be positioned or moveable to target a particular target region. The nerve modulation element may be an ultrasound transducer. The nerve modulation element may be configured to be operated at a low intensity to provide a thermal nerve block or a high intensity to effect tissue modulation.

### CROSS-REFERENCE TO RELATED APPLICATIONS

This application is a continuation of U.S. Nonprovisional patent application Ser. No. 16/524,303 , filed on Jul. 29, 2019, which is a continuation of U.S. patent application Ser. No. 14/032,553, filed Sep. 20, 2013, now U.S. Pat. No. 10,398,464, which claims the benefit of U.S. Provisional Application Ser. No. 61/704,169, filed Sep. 21, 2012, the contents of which are herein incorporated by reference in their entirety.

### TECHNICAL FIELD

The present disclosure relates to methods and apparatuses for nerve modulation techniques such as ablation of nerve tissue or other modulation techniques through the walls of blood vessels and monitoring thereof.

### BACKGROUND

Certain treatments require the temporary or permanent interruption or modification of select nerve function. One example treatment is renal nerve ablation, which is sometimes used to treat conditions related to congestive heart failure or hypertension. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves, including renal nerves, run along the walls of or in close proximity to blood vessels and thus can be accessed via the blood vessels. In some instances, it may be desirable to ablate perivascular renal nerves using ultrasound energy. The target nerves must be heated sufficiently to make them nonfunctional, but heating tissues can cause significant pain during the procedure. Pain relief during renal nerve ablation has been addressed by medication, which does not always control the pain adequately. It may be desirable to provide for alternative systems and methods for intravascular nerve modulation and pain management during the nerve modulation.

### SUMMARY

The disclosure is directed to several alternative designs, materials and methods of manufacturing medical device structures and assemblies for performing nerve ablation.

Accordingly, one illustrative embodiment is a system for nerve modulation that may include an elongate shaft having a proximal end region and a distal end region; a control unit. A first and a second transducer may be disposed on the elongate shaft adjacent to the distal end region. The first and second transducers may be electrically connected to a control unit. The first transducer may be configured to provide a thermal nerve block and the second transducer may be configured to perform nerve modulation.

Another illustrative embodiment is a method for performing intravascular nerve modulation. A nerve modulation system including an elongate shaft having a proximal end region and a distal end region may be provided. The modulation system may further include a first transducer disposed adjacent the distal end region and a second transducer disposed distal to the first transducer. The nerve modulation system may be advanced through a lumen such that the first transducer is adjacent to a first target region. A first electrical current may be supplied to the first transducer to generate a first acoustic energy and a second electrical current may be supplied to the second transducer to generate a second acoustic energy.

Another illustrative embodiment is a method for performing intravascular nerve modulation. A nerve modulation system including an elongate shaft having a proximal end region and a distal end region may be provided. The modulation system may further include a transducer disposed adjacent the distal end region.

The nerve modulation system may be advanced through a lumen such that the transducer is adjacent to a first target region. A first current may be supplied to the transducer to generate a first acoustic energy. The nerve modulation system may then be advanced distally within the lumen and a second current supplied to the transducer to generate a second acoustic energy.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 illustrates a portion of an example renal nerve modulation system.

FIG. 3 illustrates a portion of another example renal nerve modulation system.

FIG. 4 illustrates a portion of another example renal nerve modulation system.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

Certain treatments require the temporary or permanent interruption or modification of select nerve function. One example treatment is renal nerve ablation, which is sometimes used to treat conditions related to congestive heart failure or hypertension. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

While the devices and methods described herein are discussed relative to renal nerve modulation, it is contemplated that the devices and methods may be used in other treatment locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, tumor ablation, benign prostatic hyperplasia therapy, nerve excitation or blocking or ablation, modulation of muscle activity, hyperthermia or other warming of tissues, etc. In some instances, it may be desirable to ablate perivascular renal nerves with ultrasound ablation.

Ultrasound energy may be used to generate heat at a target location. The high frequency sound waves produced by an ultrasonic transducer may be directed at a target region and absorbed at the target region. As the energy emitted is absorbed, the temperature of the target region may rise. In order to perform renal nerve ablation, target nerves must be heated sufficiently to make them nonfunctional, while thermal injury to the artery wall is undesirable. Heating of the artery wall may also increase pain during the procedure. Pain relief during renal nerve ablation has been addressed by medication, which does not always control the pain adequately. A method for ablating target nerves, while providing sufficient pain management is needed. Nerve blocks for reducing pain have routinely been accomplished by anesthetics, and also by electrical stimulation. Various approaches have been used for electrical stimulation of cells, such as high frequency stimulation that is faster than the nerve refractory period so that the cells can't repolarize, or otherwise interfere with depolarization or action potential propagation. A proximal nerve block is often used to block a more distal pain.

Thermal gradients applied to axons and nerves may be used to stimulate the nerves and provide a nerve block. Stimulation of the nerves proximal to a desired ablation treatment region at a temperature sufficient for cell depolarization may “block” the nerves, thus preventing the sensation of pain during a procedure without causing irreversible thermal injury to the tissue. For example, raising the temperature approximately 5-10° C. using a variety of methods may be used to stimulate nerves to provide a thermal nerve block. It appears the temperature rise, not the absolute temperature, causes the stimulation. A low energy method may be used to create a low temperature rise proximal to the desired ablation treatment region. As used herein, the terms “low energy” or “low intensity” may be used to describe the application of energy at a power, frequency, and duration to raise the temperature (by a rise of, for example, approximately 5-10° C.) of a target region to a level sufficient to prevent the sensation of pain during a procedure. In contrast, as used herein the terms “high energy” or “high intensity” may be used to describe the application of energy at a power, frequency, and duration to raise the temperature of a target region to at least a temperature at which tissue begins to denature or cause irreversible tissue changes (for example, at least 50° C.). It is contemplated that the power level, frequency and/or duration of the energy application may be adjusted to achieve the desired temperature rise. Methods of heating may include applying energy using lasers or other light, with or without a dye, focused ultrasound, electrical current, magnetically induced current, or direct heating with a heated probe, balloon, or infusion, etc. In some instances, the temperatures induced by these methods may be too low to cause significant thermal injury for short exposures to local tissues, but may be used to stimulate a nerve.

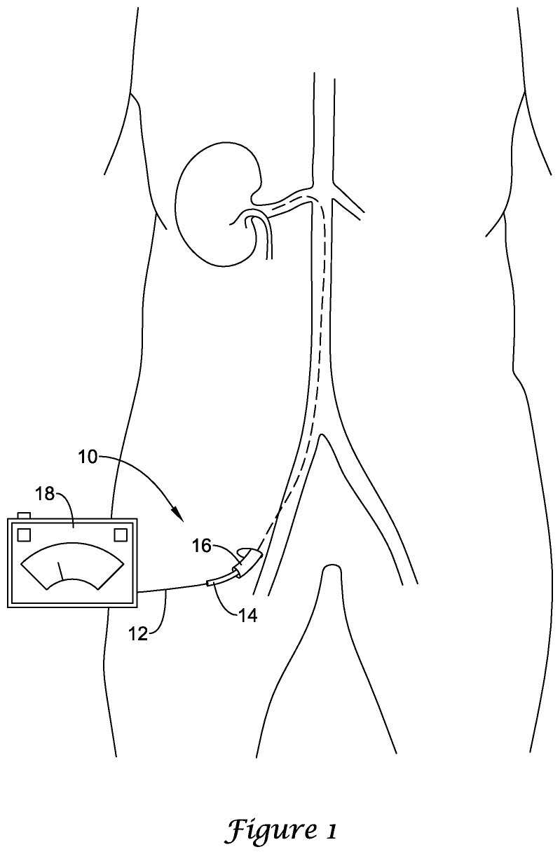

FIG. 1 is a schematic view of an illustrative renal nerve modulation system 10 in situ. System 10 may include an element 12 for providing power to a transducer disposed adjacent to, about, and/or within a central elongate shaft 14 and, optionally, within a sheath 16, the details of which can be better seen in subsequent figures. A proximal end of element 12 may be connected to a control and power element 18, which supplies the necessary electrical energy to activate the one or more transducers at or near a distal end of the element 12. The control and power element 18 may include monitoring elements to monitor parameters such as power, temperature, voltage, and/or frequency and other suitable parameters as well as suitable controls for performing the desired procedure. In some instances, the power element 18 may control an ultrasound transducer. The transducer may be configured to operate at a frequency of approximately 9-10 megahertz (MHz), for example. It is contemplated that any desired frequency may be used, for example, from 1-20 MHz. However, it is contemplated that frequencies outside this range may also be used, as desired. While the term “ultrasound” is used herein, this is not meant to limit the range of vibration frequencies contemplated.

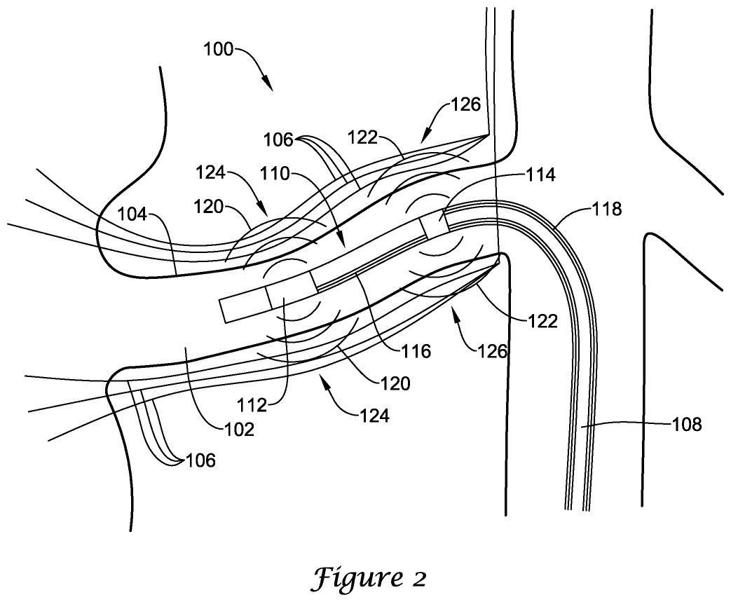

FIG. 2 is an illustrative embodiment of a distal end of a renal nerve modulation system 100 disposed within a body lumen 102 having a vessel wall 104. The vessel wall 104 may be surrounded by local body tissue. The local body tissue may comprise adventitia and connective tissues, nerves, fat, fluid, etc. in addition to the muscular vessel wall 104. For example, the local body tissue may include one or more nerves, ganglia, etc. 106 disposed adjacent the vessel wall 104. A portion of the tissue may be the desired treatment region 124 and a portion of the tissue may be the desired pain blocking region. It is contemplated that there may be any number of treatment regions, sub-regions within the target treatment region 124, or any number of regions for providing pain management.

The system 100 may include an elongate shaft 108 having a distal end region 110. The elongate shaft 108 may extend proximally from the distal end region 110 to a proximal end configured to remain outside of a patient's body. The proximal end of the elongate shaft 108 may include a hub attached thereto for connecting other treatment devices or providing a port for facilitating other treatments. It is contemplated that the stiffness of the elongate shaft 108 may be modified to form a modulation system 100 for use in various vessel diameters and various locations within the vascular tree. The elongate shaft 108 may further include one or more lumens extending therethrough. For example, the elongate shaft 108 may include a guidewire lumen and/or one or more auxiliary lumens. The lumens may have a variety of configurations and/or arrangements. For example, the guidewire lumen may extend the entire length of the elongate shaft 108 such as in an over-the-wire catheter or may extend only along a distal portion of the elongate shaft 108 such as in a single operator exchange (SOE) catheter. These examples are not intended to be limiting, but rather examples of some possible configurations. While not explicitly shown, the modulation system 100 may further include temperature sensors/wire, an infusion lumen, radiopaque marker bands, fixed guidewire tip, a guidewire lumen, external sheath and/or other components to facilitate the use and advancement of the system 100 within the vasculature. It is further contemplated that the modulation system 100 may include one or more centering baskets, expandable framework, and/or expandable balloons to center or otherwise position the modulation system 100 within the body lumen 102.

The system 100 may include a distal ablation transducer 112 positioned adjacent the distal end region 110 of the elongate shaft. While the ablation transducer 112 is described as an ultrasonic transducer, it is contemplated that other methods and devices for raising the temperature of the nerves may be used, such as, but not limited to: radiofrequency, microwave, or other acoustic, optical, electrical current, direct contact heating, or other heating. The ablation transducer 112 may be formed from any suitable material such as, but not limited to, lead zirconate titanate (PZT). It is contemplated that other ceramic or piezoelectric materials may also be used. While not explicitly shown, the ablation transducer 112 may have a first radiating surface, a second radiating surface, and a perimeter surface extending around the outer edge of the ablation transducer 112. In some instances, the ablation transducer 112 may include a layer of gold, or other conductive layer, disposed on the first and/or second side over the PZT crystal for connecting electrical leads, such as lead 116, to the ablation transducer 112. In some embodiments, the ablation transducer 112 may be structured to radiate acoustic energy from a single radiating surface. In such an instance, one radiating surface may include a backing layer to direct the acoustic energy in a single direction. In other embodiments, the ablation transducer 112 may be structured to radiate acoustic energy from two radiating surfaces. In some instances, one or more tie layers may be used to bond the gold to the PZT. For example, a layer of chrome may be disposed between the PZT and the gold to improve adhesion. In other instances, the transducers 112, 114 may include a layer of chrome over the PZT followed by a layer of nickel, and finally a layer of gold. These are just examples. It is contemplated that the layers may be deposited on the PZT using sputter coating, although other deposition techniques may be used as desired.

It is contemplated that the radiating surface (surface which radiates acoustic energy) of the ablation transducer 112 may take any shape desired, such as, but not limited to, square, rectangular, polygonal, circular, oblong, cylindrical, etc. The acoustic energy from the radiating surface of the ablation transducer 112 may be transmitted in a spatial pressure distribution related to the shape of the ablation transducer 112. With exposures of appropriate power and duration, lesions formed during ablation may take a shape similar to the contours of the pressure distribution. As used herein, a “lesion” may be a change in tissue structure or function due to injury (e.g. tissue damage caused by the ultrasound). Thus, the shape and dimensions of the ablation transducer 112 may be selected based on the desired treatment and the shape best suited for that treatment. It is contemplated that the ablation transducer 112 may also be sized according to the desired treatment region. For example, in renal applications, the ablation transducer 112 may be sized to be compatible with a 6 French guide catheter, although this is not required.

In some embodiments, the ablation transducer 112 may be formed of a separate structure and attached to the elongate shaft 108. For example, the ablation transducer 112 may be bonded or otherwise attached to the elongate shaft 108. In some instances, the ablation transducer 112 may include a ring or other retaining or holding mechanism (not explicitly shown) disposed around the perimeter of the ablation transducer 112 to facilitate attachment of the ablation transducer 112. The ablation transducer 112 may further include a post, or other like mechanism, affixed to the ring such that the post may be attached to the elongate shaft 108 or other member. In some instances, the rings may be attached to the ablation transducer 112 with a flexible adhesive, such as, but not limited to, silicone. However, it is contemplated that the rings may be attached to the ablation transducer 112 in any manner desired. While not explicitly shown, in some instances, the elongate shaft 108 may be formed with grooves or recesses in an outer surface thereof. The recesses may be sized and shaped to receive the ablation transducer 112. For example, the ablation transducer 112 may be disposed within the recess such that a first radiating surface contacts the outer surface of the elongate shaft 108 and a second radiating surface is directed towards a desired treatment region. However, it is contemplated that the ablation transducer 112 may be affixed to the elongate shaft in any manner desired.

In some embodiments, the ablation transducer 112 may be affixed to an outer surface of the elongate shaft 108 such that the surface of the ablation transducer 112 is exposed to blood flow through the vessel. As the power is relayed to the ablation transducer 112, the power that does not go into generating acoustic power generates heat. As the ablation transducer 112 heats, it becomes less efficient, thus generating more heat. Passive cooling provided by the flow of blood may help improve the efficiency of the ablation transducer 112. As such, additional cooling mechanisms may not be necessary. However, in some instances, additional cooling may be provided by introducing a cooling fluid to the modulation system.

In some instances, the ablation transducer 112 may comprise a plurality of transducers. For example, in some embodiments, the ablation transducer 112 may include a number of transducers (two, three, four, or more) spaced about the circumference of the elongate shaft 108. This may allow for ablation of multiple radial locations about the body lumen 102 simultaneously. In other embodiments, the ablation transducer 112 may comprise a focused or phased array of transducers. The array may be configured to be directed at a focus region such that multiple transducers are radiating energy at a common target region. It is further contemplated that the ablation transducer 112 may comprise two or more longitudinally spaced transducers.

The distal ablation transducer 112 may be connected to a control unit (such as control unit 18 in FIG. 1) by electrical conductor(s) 116. In some embodiments, the electrical conductor(s) may be disposed within a lumen of the elongate shaft 108. In other embodiments, the electrical conductor(s) 116 may extend along an outside surface of the elongate shaft 108. The electrical conductor(s) 116 may provide electricity to the ablation transducer 112 which may then be converted into acoustic energy 120. The acoustic energy 120 may be directed from the ablation transducer 112 in a direction generally perpendicular to the radiating surfaces of the ablation transducer 112. As discussed above, acoustic energy radiates from the ablation transducer 112 in a pattern related to the shape of the transducer 112 and lesions formed during ablation take shape similar to contours of the pressure distribution.

The modulation system 100 may further include a proximal stimulation transducer 114 positioned proximal to the ablation transducer 112. It is contemplated that the ablation transducer 112 and the stimulation transducer 114 may be placed at any longitudinal location along the elongate shaft 108 desired. In some embodiments, the stimulation transducer 114 may be an ultrasound transducer similar in form and function to the ablation transducer 112 discussed above. However, the stimulation transducer 114 may be operated at a lower intensity relative to the ablation transducer 112 to stimulate the adjacent nerves in a location 126 proximal to the desired treatment region 124 to block pain signals traveling to the brain. Power may be supplied to the stimulation transducer 114 through an electrical conductor 118 such that the surrounding nerves are heated approximately 5-10° C. greater than the surrounding body temperature. Thus, the surrounding nerves are heated to approximately 40-49° C., depending on the body temperature of the patient. However, these ranges are merely exemplary and the thermal block may be performed at temperature outside these ranges. However, it is contemplated that the location 126 at which the thermal nerve block is performed should not be heated to the point at which tissue begins to denature or irreversibly change, for example, approximately 50-60° C.

Distal tissue ablation may cause progressive nerve damage which may cause continued pain sensation (likely a general or referred pain rather than via nociceptors) which is transmitted proximally through the nerves. Stimulation of the nerves at a location 126 proximal to the ablation target region 124 may block these pain sensations from reaching the brain. After the ablation is completed, the proximal stimulation can be stopped immediately or after a short time. If there is mild pain after the ablation procedure, conventional medications can be used to relieve any residual pain.

While the stimulation transducer 114 is described as an ultrasonic transducer, it is contemplated that other methods and devices for raising the temperature of the nerves may be used, such as, but not limited to: microwave, or other acoustic, optical, electrical current, direct contact heating, or other mild heating. The use of the word transducer is not intended to limit the device to an ultrasound stimulation transducer; rather the transducer 114 may be a microwave element, a radiofrequency electrode, or other means for raising the temperature of the nerves. The nerve block may result from the temperature gradient stimulating the nerve. Thus, as the nerve cools, repeat stimulation of the nerve(s) may be required to block the pain. In some instances, the thermal pain block can be ramped up slowly (such as over a time period of seconds) or modulated in frequency, amplitude, and duration or duty cycle to minimize the sensation of pain. While the thermal nerve block is described herein are discussed relative to ultrasound tissue modulation, it is contemplated that the devices and methods may be used in other applications, such as, but not limited to: radiofrequency (RF) ablation, laser or microwave or other thermal ablation, cryothermal ablation, or with chemoablation, to similarly reduce periprocedural pain.

In some embodiments, the stimulation transducer 114 may be pulsed to cause repeated depolarization of the nerve. For example, the stimulation transducer 114 may be pulsed at frequencies of 100 Hz and greater. The thermal block may be accomplished by a succession of temperature gradients. If the heat is focused for example by focused ultrasound, to a small region, repeat depolarization may be done rapidly to enable a nerve block. Pulsed low intensity heating may block the nerve while preserving the nerve by maintaining the average temperature of the target tissue below 50° C. However, in some instances, pulsed low intensity heating may raise the temperature of the target tissue above 50° C. very briefly such that irreversible tissue damage does not occur.

In some instances, the stimulation transducer 114 may comprise a plurality of transducers. For example, in some embodiments, the stimulation transducer 114 may include a number of transducers (two, three, four, or more) spaced about the circumference of the elongate shaft 108. This may allow for stimulation of multiple radial locations about the body lumen 102 simultaneously. In other embodiments, the stimulation transducer 114 may comprise a focused or phased array of transducers. The array may be configured to be directed at a focus region such that multiple transducers are radiating energy at a common target region. It is further contemplated that the stimulation transducer 114 may comprise two or more longitudinally spaced transducers.

The proximal stimulation transducer 114 may be connected to a control unit (such as control unit 18 in FIG. 1) by electrical conductor(s) 118. In some embodiments, the electrical conductor(s) may be disposed within a lumen of the elongate shaft 108. In other embodiments, the electrical conductor(s) 118 may extend along an outside surface of the elongate shaft 108. The electrical conductor(s) 118 may provide electricity to the stimulation transducer 114 which may then be converted into acoustic energy 122. The acoustic energy 122 may be directed from the stimulation transducer 114 in a direction generally perpendicular to the radiating surfaces of the stimulation transducer 114.

The modulation system 100 may be advanced through the vasculature in any manner known in the art. For example, system 100 may include a guidewire lumen to allow the system 100 to be advanced over a previously located guidewire. In some embodiments, the modulation system 100 may be advanced, or partially advanced, within a guide sheath such as the sheath 16 shown in FIG. 1. Once the ablation transducer 112 of the modulation system 100 has been placed adjacent to the desired treatment area 124, positioning mechanisms may be deployed, such as centering baskets, if so provided. While not explicitly shown, the ablation transducer 112 and the stimulation transducer 114 may be connected to a single control unit or to separate control units (such as control unit 18 in FIG. 1) by electrical conductors 116, 118. Once the modulation system 100 has been advanced to the treatment region, energy may be supplied to the ablation transducer 112 and the stimulation transducer 114. In some instances, energy may be supplied first to the stimulation transducer 114 such that the thermal nerve block may be commenced prior to beginning tissue ablation. Energy may continue to be supplied to the stimulation transducer 114 during the entire ablation procedure. It is further contemplated that energy may continue to be supplied after completing the tissue ablation for further pain management. In some instances, pain medication may be used in addition to the thermal nerve block in order to provide an increased analgesic effect. In some instances, the thermal nerve block may increase the effectiveness of analgesic medication, while in other instances; the analgesic medication may increase the effectiveness of the thermal block. Energy may be supplied to both the ablation transducer 112 and the stimulation transducer 114 simultaneously or in an alternating fashion at desired. The amount of energy delivered to the ablation transducer 112 may be determined by the desired treatment as well as the feedback provided by the system 100. It is contemplated that energy may be supplied to the stimulation transducer 114 such that the thermal pain block can be ramped up slowly (such as over a time period of seconds) or modulated in frequency, amplitude, and duration or duty cycle to minimize the sensation of pain.

In some instances, the elongate shaft 108 may be rotated and additional ablation can be performed at multiple locations around the circumference of the vessel 102. In some instances, a slow automated “rotisserie” rotation can be used to work around the circumference of the vessel 102, or a faster spinning can be used to simultaneously ablate around the entire circumference. The spinning can be accomplished with a micro-motor or by spinning a drive shaft. In some embodiments, ultrasound sensor information can be used to selectively turn on and off the ablation transducer 112 to warm any cool spots or accommodate for veins, or other tissue variations. The number of times the elongate shaft 108 is rotated at a given longitudinal location may be determined by the number and size of the ablation transducer 112 on the elongate shaft 108. Once a particular location has been ablated, it may be desirable to perform further ablation procedures at different longitudinal locations. Once the elongate shaft 108 has been longitudinally repositioned, energy may once again be delivered to the ablation transducer 112 and the stimulation transducer 114. If necessary, the elongate shaft 108 may be rotated to perform ablation around the circumference of the vessel 102 at each longitudinal location. This process may be repeated at any number of longitudinal locations desired. It is contemplated that in some embodiments, the system 100 may include ablation transducers and/or stimulation transducers at various positions along the length of the modulation system 100 such that a larger region may be treated without longitudinal displacement of the elongate shaft 108.

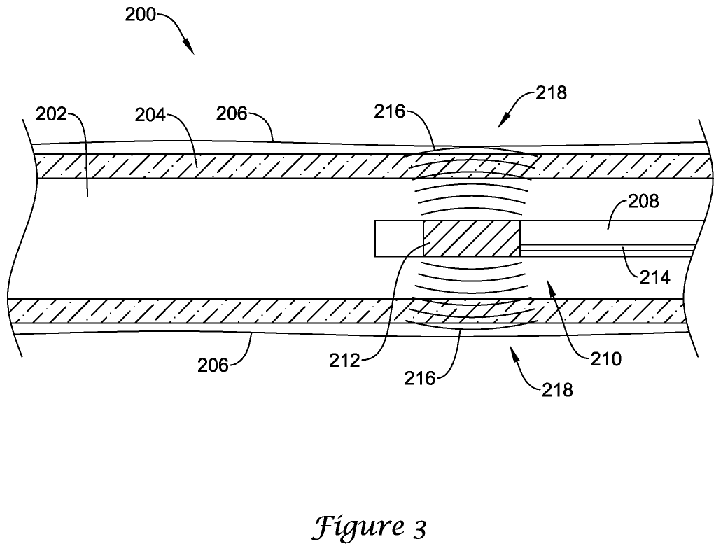

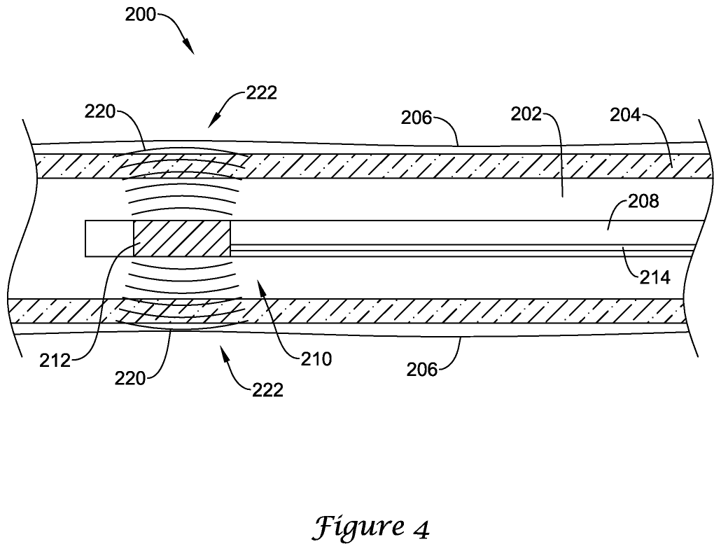

FIG. 3 is an illustrative embodiment of a distal end of a renal nerve modulation system 200 that may be similar in form and function to other systems disclosed herein. The modulation system 200 may be disposed within a body lumen 202 having a vessel wall 204. The vessel wall 204 may be surrounded by local body tissue. The local body tissue may comprise adventitia and connective tissues, nerves, fat, fluid, etc. in addition to the muscular vessel wall 204. For example, the local body tissue may include one or more nerves, ganglia, etc. 206 disposed adjacent the vessel wall 204. A portion of the tissue may be the desired treatment region 222 (shown in FIG. 4) and a portion of the tissue may be the desired pain blocking region 218. It is contemplated that there may be any number of treatment regions, sub-regions within the target treatment region 222, or any number of regions for providing pain management.

The system 200 may include an elongate shaft 208 having a distal end region 210. The elongate shaft 208 may extend proximally from the distal end region 210 to a proximal end configured to remain outside of a patient's body. The proximal end of the elongate shaft 208 may include a hub attached thereto for connecting other treatment devices or providing a port for facilitating other treatments. It is contemplated that the stiffness of the elongate shaft 208 may be modified to form a modulation system 200 for use in various vessel diameters and various locations within the vascular tree. The elongate shaft 208 may further include one or more lumens extending therethrough. For example, the elongate shaft 208 may include a guidewire lumen and/or one or more auxiliary lumens. The lumens may be configured in any way known in the art. While not explicitly shown, the modulation system 200 may further include temperature sensors/wire, an infusion lumen, radiopaque marker bands, fixed guidewire tip, a guidewire lumen, external sheath and/or other components to facilitate the use and advancement of the system 200 within the vasculature.

The system 200 may include an ultrasound transducer 212 positioned adjacent the distal end region 210 of the elongate shaft 208. The ultrasound transducer 212 may be configured to be operated at a low intensity to provide a thermal nerve block and at a higher intensity to perform tissue modulation. The ultrasound transducer 212 may be formed from any suitable material such as, but not limited to, lead zirconate titanate (PZT). It is contemplated that other ceramic or piezoelectric materials may also be used. It is contemplated that the transducer 212 may have similar form and function to the transducers discussed above. In some instances, the transducer 212 may comprise a plurality of transducers. For example, in some embodiments, the transducer 212 may include a number of transducers (two, three, four, or more) spaced about the circumference of the elongate shaft 208. This may allow for ablation of multiple radial locations about the body lumen 202 simultaneously. In other embodiments, the transducer 212 may comprise a focused or phased array of transducers. The array may be configured to be directed at a focus region such that multiple transducers are radiating energy at a common target region. It is further contemplated that the transducer 212 may comprise two or more longitudinally spaced transducers.

The transducer 212 may be connected to a control unit (such as control unit 18 in FIG. 1) by electrical conductor(s) 214. In some embodiments, the electrical conductor(s) 214 may be disposed within a lumen of the elongate shaft 208. In other embodiments, the electrical conductor(s) 214 may extend along an outside surface of the elongate shaft 208. The electrical conductor(s) 214 may provide electricity to the transducer 212 which may then be converted into acoustic energy 216. The acoustic energy 216 may be directed from the transducer 212 in a direction generally perpendicular to the radiating surfaces of the transducer 212. As discussed above, acoustic energy radiates from the ablation transducer 212 in a pattern related to the shape of the transducer 212 and lesions formed during ablation take shape similar to contours of the pressure distribution.

The modulation system 200 may be advanced through the vasculature in any manner known in the art. For example, system 200 may include a guidewire lumen to allow the system 200 to be advanced over a previously located guidewire. In some embodiments, the modulation system 200 may be advanced, or partially advanced, within a guide sheath such as the sheath 16 shown in FIG. 1. Once the transducer 212 of the modulation system 200 has been placed adjacent to the first region 218, positioning mechanisms may be deployed, such as centering baskets, if so provided. Once the modulation system 200 has been advanced to the first region 218, energy may be supplied to the transducer 212 at a low intensity such that the surrounding nerves are heated approximately 5-10° C. greater than the surrounding body temperature. This may stimulate the nerves to prevent pain signals from passing to the brain during tissue ablation. It is contemplated that energy may be supplied to the transducer 212 such that the thermal pain block can be ramped up slowly (such as over a time period of seconds) or modulated in frequency, amplitude, and duration or duty cycle to minimize the sensation of pain. Once the surrounding to nerves have been heated sufficiently to block pain signals, the modulation system may be advanced distally within the body lumen 202 to a second treatment region 222, as shown in FIG. 4. In some instances, pain medication may be used in addition to the thermal nerve block in order to provide an increased analgesic effect. In some instances, the thermal nerve block may increase the effectiveness of analgesic medication, while in other instances; the analgesic medication may increase the effectiveness of the thermal block. It is further contemplated that in some instances, the modulation system 200 may be required to be repositioned adjacent to the first region 218 to repeatedly heat the surrounding tissue to provide a continuous thermal nerve block, depending on the duration of the desired treatment.

Once the modulation system 200 has been advanced to the second region 222, energy may again be supplied to the transducer 212 such that energy 220 is directed from the transducer 212 at a higher intensity to perform tissue modulation. The amount of energy delivered to the ablation transducer 212 may be determined by the desired treatment as well as the feedback provided by the system 200. In some instances, the elongate shaft 208 may be rotated and additional ablation can be performed at multiple locations around the circumference of the vessel 202. In some instances, a slow automated “rotisserie” rotation can be used to work around the circumference of the vessel 202, or a faster spinning can be used to simultaneously ablate around the entire circumference. The spinning can be accomplished with a micro-motor or by spinning a drive shaft. In some embodiments, ultrasound sensor information can be used to selectively turn on and off the transducer 212 to warm any cool spots or accommodate for veins, or other tissue variations. The number of times the elongate shaft 208 is rotated at a given longitudinal location may be determined by the number and size of the transducer(s) 212 on the elongate shaft 208. Once a particular location has been ablated, it may be desirable to perform further ablation procedures at different longitudinal locations. Once the elongate shaft 208 has been longitudinally repositioned, energy may once again be delivered to the transducer 212. If necessary, the elongate shaft 208 may be rotated to perform ablation around the circumference of the vessel 202 at each longitudinal location. This process may be repeated at any number of longitudinal locations desired. It is contemplated that in some embodiments, the system 200 may include transducers at various positions along the length of the modulation system 200 such that a larger region may be treated without longitudinal displacement of the elongate shaft 208.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A method for performing intravascular nerve modulation, the method comprising:
advancing a nerve modulation system that comprises an elongate shaft having a proximal end region and a distal end region, a first transducer disposed adjacent the distal end region, and a second transducer disposed adjacent to the first transducer through a body lumen of a patient such that the first transducer is adjacent to a first target region;
operating the first transducer to generate a first energy that is directed to the first target region such that a temperature of the first target region is raised to a first temperature that is sufficient to generate a thermal nerve block; and
operating the second transducer to generate a second energy different from the first energy, said second energy directed to a second target region adjacent to the second transducer such that a temperature of the second target region is raised to a second temperature that is greater than the first temperature and is sufficient to perform tissue modulation.

2. The method of claim 1, wherein the first energy is selected from acoustic energy, optical energy, electrical energy and direct contact heating.

3. The method of claim 1, wherein the second energy is selected from acoustic energy, optical energy, electrical energy and direct contact heating.

4. The method of claim 1, wherein the first energy is repeatly generated to maintain the thermal nerve block.

5. The method of claim 1, wherein the thermal pain block is ramped up over a time to minimize the sensation of pain.

6. The method of claim 1, wherein the thermal nerve block is modulated in frequency, amplitude, and duration or duty cycle to minimize the sensation of pain.

7. The method of claim 1, wherein the tissue modulation comprises nerve modulation.

8. The method of claim 1, wherein the first transducer is selected from an ultrasound transducer, a radiofrequency electrode, a microwave element, and a laser.

9. The method of claim 1, wherein the second transducer is selected from an ultrasound transducer, a radiofrequency electrode, a microwave element, and a laser.

10. The method of claim 1, wherein the first energy raises a temperature of the first target region by 5-10° C. to generate a thermal nerve block.

11. The method of claim 1, wherein the first first transducer is operated prior to operating the second transducer.

12. The method of claim 1, wherein the first and second tranducers are operated simultaneously.

13. The method of claim 1, wherein the first transducer is positioned proximal to the second transducer.

14. A tissue modulation system, comprising:
an elongate shaft having a distal region;
a high energy tissue modulation member coupled to the distal region;
a low energy tissue modulation member coupled to the distal region and positioned adjacent to the high energy tissue modulation member; and
a control unit.

15. The tissue modulation system of claim 14, further comprising a control unit.

16. The tissue modulation system of claim 15, wherein the control unit supplies power to the high energy tissue modulation member and the low energy tissue modulation member alternately.

17. The tissue modulation system of claim 15, wherein the control unit supplies power to the high energy tissue modulation member and the low energy tissue modulation member simultaneously.

18. The tissue modulation system of claim 14, wherein the high energy tissue modulation member comprises a plurality of transducers.

19. The tissue modulation system of claim 14, wherein the low energy tissue modulation member comprises a plurality of transducers.

20. The tissue modulation system of claim 14, wherein low energy tissue modulation member is selected from an ultrasound transducer, a radiofrequency electrode, a microwave element, and a laser and wherein high energy tissue modulation member is selected from an ultrasound transducer, a radiofrequency electrode, a microwave element, and a laser.
